(19) United States
(12) Patent Application Publication
(10) Pub. No.: US 2024/0377098 A1
(43) Pub. Date: Nov. 14, 2024

(54) BUILDING VENTILATION SYSTEM

(71) Applicant: James Edwin DALLY, Kalamazoo, MI (US)

(72) Inventor: James Edwin DALLY, Kalamazoo, MI (US)

(21) Appl. No.: 18/642,650

(22) Filed: Apr. 22, 2024

Related U.S. Application Data
(63) Continuation-in-part of application No. 18/196,254, filed on May 11, 2023, now Pat. No. 11,964,541.

(51) Int. Cl. F24F 13/068; B60H 1/00; B60H 1/34

(52) U.S. Cl. CPC F24F 13/068; B60H 1/00371; B60H 1/3407

(57) ABSTRACT

A ventilation system for an interior space includes an air handling unit that moves air through the interior space and a return duct extending therefrom and having a return opening proximate to a floor of the interior space. The ventilation system also includes at least one perforated panel arranged beneath a ceiling of the interior space and a plenum defined between the ceiling and the at least one perforated panel, wherein the supply air outlet is in communication with the plenum to distribute air within the plenum and pressurize the plenum, such that air flow is induced through the at least one perforated panel as a high volume and low turbulence blanket of air that descends through the interior space and is received by the return opening in the return duct for recirculation through the air handling unit.

### CROSS-REFERENCE TO RELATED APPLIATIONS

This application is a continuation in part of U.S. patent application Ser. No. 18/196,254, filed May 11, 2023, the contents of which are hereby incorporated by reference in their entirety.

### FIELD OF THE DISCLOSURE

The present disclosure relates generally to ventilation systems, and more particularly, to ventilation systems for enclosed building structures.

### BACKGROUND OF THE DISCLOSURE

In the wake of the Covid-19 pandemic, there is an impetus to improve air quality and ventilation in indoor spaces where groups of people tend to congregate, such as within buildings. Further, people spend a significant amount of their lives indoors, but poor indoor air quality may cause adverse health effects on the occupants, including sick building syndrome (SBS), allergic rhinitis, and hypersensitivity pneumonitis. Studies have shown that exposure to pollutants such as carbon dioxide is linked to significant decrements to decision-making, and air quality directly impacts the health and the productivity of a building's occupants and constitutes a health hazard. Indeed, the World Health Organization estimates that 12.7% of deaths could be avoided by improving the indoor environment and ambient air quality around the world. Accordingly, air quality is an important consideration of a building's indoor environment.

Conventionally, building heating and ventilation systems replace air within an interior space with conditioned air at high velocity to blow the conditioned air into the space and intentionally stir up the air to balance and evenly distribute air temperature. As used herein, the term “conditioned air” may be defined as air that has been treated in ways such as circulating, heating, cooling, humidifying, dehumidifying, sterilizing, ionizing, filtering, augmenting or replacing with exterior air, any combination thereof, or the like. While operable to provide thermally comfortable temperatures, these high velocity air forcing distribution methods also mix and spread pathogens, which can negatively impact occupant health.

Accordingly, a need exists for a building ventilation system that does not agitate the interior air and distribute pathogens, but that still circulates and replaces the air with conditioned air that may be heated, cooled, purified (filtered), and/or sterilized as the need may be.

### BRIEF DESCRIPTION OF THE DRAWINGS

The accompanying drawings, which are incorporated into and constitute a part of this specification, illustrate one or more examples of embodiments and, together with the description of example embodiments, serve to explain the principles and implementations of the embodiments.

FIG. 1 is a schematic diagram of a multi-passenger vehicle ventilation system for use in a vehicle such as a school bus in accordance with certain embodiments;

FIG. 2 is a schematic diagram of a perforated panel in accordance with certain embodiments;

FIG. 3 is a cut-away rear view of a bus with a ventilation system in accordance with certain embodiments;

FIG. 4 is a partial cut-away front view of a bus with a ventilation system in accordance with certain embodiments;

FIG. 5 is an isometric view of an air processing unit (APU) in accordance with certain embodiments;

FIG. 6 is an isometric view of an APU equipped with a heat exchange unit in accordance with certain embodiments;

FIG. 7 is a schematic diagram of a ventilation system for use in an interior space, such as in a building, according to one or more embodiments;

FIG. 8 schematically depicts a variation of the ventilation system of FIG. 7, according to one or more alternate embodiments;

FIG. 9 schematically depicts the ventilation system of FIG. 7 integrated within an elevator carriage, according to one or more embodiments; and

FIG. 10 schematically depicts the ventilation system of FIG. 7 integrated within an area surrounding a store checkout counter, according to one or more alternate embodiments.

### DESCRIPTION OF EXAMPLE EMBODIMENTS

Embodiments of the present disclosure will now be described in detail with reference to the accompanying Figures. Like elements in the various figures may be denoted by like reference numerals for consistency. Further, in the following detailed description of embodiments of the present disclosure, numerous specific details are set forth in order to provide a more thorough understanding of the claimed subject matter. However, it will be apparent to one of ordinary skill in the art that the embodiments disclosed herein may be practiced without these specific details. In other instances, well-known features have not been described in detail to avoid unnecessarily complicating the description. Additionally, it will be apparent to one of ordinary skill in the art that the scale of the elements presented in the accompanying Figures may vary without departing from the scope of the present disclosure.

The embodiments described herein are directed to vehicle ventilation systems, such as multi-passenger vehicle ventilation systems, and building ventilation systems. Embodiments disclosed herein also describe ventilation systems for confined spaces, such as elevator carriages or store check-out aisles. The following description is illustrative only and is not intended to be in any way limiting. Other embodiments will readily suggest themselves to those of ordinary skill in the art having the benefit of this disclosure. Reference will be made in detail to implementations of the example embodiments as illustrated in the accompanying drawings.

In the description of example embodiments that follows, references to “one embodiment”, “an embodiment”, “an example embodiment”, “certain embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. The term “exemplary” when used herein means “serving as an example, instance or illustration.” Any embodiment described herein as “exemplary” is not necessarily to be construed as preferred or advantageous over other embodiments.

In the interest of clarity, not all of the routine features of the implementations described herein are shown and described. It will be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

Described herein is a “High Volume Low Turbulence” (HVLT) system for delivering air through a large plenum (one or more plenums or sub-plenums are contemplated) across substantially the entire top of a vehicle cabin's ceiling through an array of perforations. Also described herein is a HVLT system for delivering air through a plenum within a room or space in a building, within an elevator carriage, or above a check-out aisle. This approach allows the air, which is evenly pressurized within the plenum(s), to fall planarly, like a sheet across the entire space beneath the plenum, consistently and in blanket fashion.

In certain embodiments, “Air Processing Units” (APUs) are used to supply air. The air may be conditioned as appropriate for passenger/occupant comfort and safety, and such conditioning may include any one or more of heating, cooling, humidifying, dehumidifying, sterilizing, ionizing, filtering, augmenting or replacing with exterior air, or the like. The term “conditioning” will thus be used herein to denote any one or more of these actions.

In certain embodiments, two or more APU's are deployed in a multi-passenger vehicle or an interior space of a building. Reasons for this include 1) It allows the air plenum(s) to be equally pressurized from both sides, 2) it provides redundancy should one of the system's equipment fail, 3) It provides the most efficient and accurate air cycling of the cabin/interior space, and 4) It allows electric heating and air conditioning and other conditioning to be added via a module.

The APU entails a cabinet with a tamper proof access door which is equipped with a battle switch or circuit interrupter switch for safety of maintenance crew due to the dangers of exposure to Ultraviolet-C (UVC) lighting which may be used for sterilization. The APU operates like a black out box so that there is no possibility of occupants being exposed to the UVC lighting. The APU is sized for the maximum amount of air exposure to the sterilizing UVC bulbs when used, and the bulbs may be designed to special wavelengths of nanometers depending on the targeted pathogens, such as Covid-19. The APU may be designed to expose the air in ratio to the proper UVC lighting in order to implement an effective sterilization regime. The APU can be equipped with both heating and cooling, or any conditioning equipment as that term is defined above.

In certain embodiments, there is a supply duct that comes off each APU and runs up a sidewall of the vehicle cabin and then runs down the side of the of the vehicle, on top of the windows to feed the plenum(s). In certain embodiments, this duct will be plastic so that it is extremely durable and blends with the interior decor of most multi-passenger vehicle cabins. Small in-line booster fans may be deployed within this ductwork to ensure that sub-plenums, when present, remain equally pressurized in a balanced manner.

In certain embodiments, a fully perforated ceiling panel may be implemented, to provide a high volume and low turbulent sheet of air that blankets down across the passengers in a substantially vertical route (plane), carrying particulates down with it and substantially preventing them from dispersing laterally and spreading among passengers. This ceiling panel may have thousands of substantially equally spaced holes (apertures) that allow an even, yet slowly, controlled discharge of air. The plenum depth above the panel ranges from about one to three inches depending on the cabin height for instance.

In certain embodiments, a liquid ceramic heat reflective coating may be applied, as well as a liquid sound reducing coating on the inside bottom of the cabin roof skin.

The pressurized air plenum may be segmented into several “zoned” smaller sub-plenums with the use of small air dampers that allow controllable, even, balanced pressurization. Each sub-plenum may be balanced with manual dampers from the supply duct(s). This blanket of air then falls and is pulled into return air ducts that are located at the perimeter of each seat causing the wind to act like a shear. The air is then returned via a plastic ducting system that is mounted along the floor at both sides of the cabin with branch extensions that feed return ducts under the seats. The seats are equipped with return air ducts at the front, rear, and aisle end. All of this permits the air to be pulled down vertically directly in all areas especially in front of the occupants, which vastly reduces lateral air motion and eliminates pathogen transfer throughout the air system of the cabin.

In certain embodiments, and for example depending on the geographic location of the vehicle, heat and/or air conditioning may be provided. In certain designs, for example in retrofit configurations of existing passenger vehicles, existing box heaters may be removed as they may cause undesired mixing of air that disrupts the blanket of air approach. In certain embodiments the existing heating element can be installed in an additional module attached to the APU as further detailed below.

In certain embodiments, an air screen or air door is located over the vehicle (e.g. school bus) main access door. This screen/air door can automatically come on (activate) upon the access door being opened. Advantages of such an arrangement are: 1) It prevents the loss of cooled or heated (or otherwise conditioned) air, thus increasing efficiency, 2) It prevents outside contamination from entering the cabin, and 3) It prevents insects from entering the cabin.

It will be appreciated that while discussed primarily in terms of a school bus, the system is applicable to any type of multi-passenger vehicle cabin including, but not limited to, public transportation buses, aeronautical aircraft, tour buses, 15-passenger vans or the like, trains, subways, and so on. Moreover, as discussed in more detail below, the systems described herein may also be employed in a building setting or in any location where air ventilation in accordance with the present disclosure is desired.

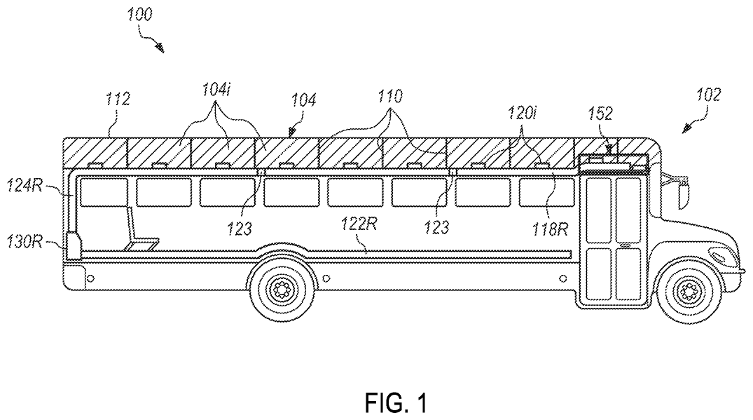

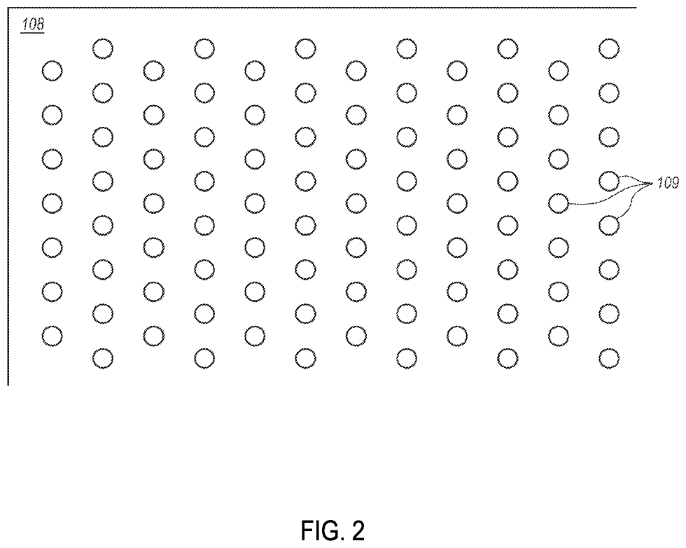

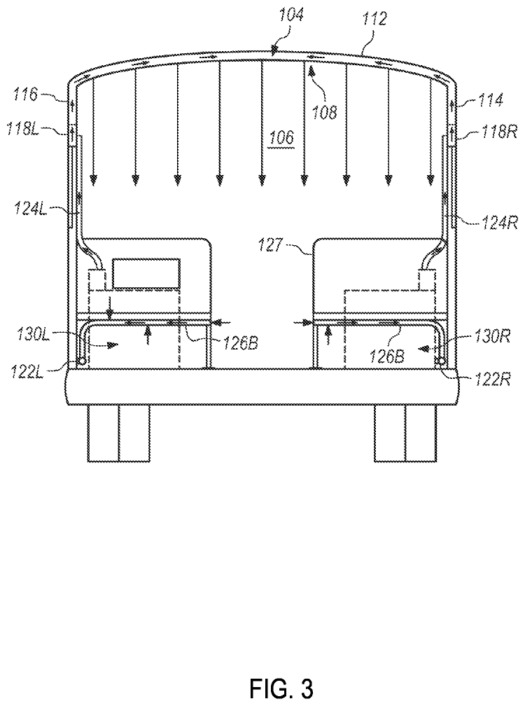

FIG. 1 schematically depicts a multi-passenger vehicle ventilation system 100 for use in a vehicle, such as a school bus 102, in accordance with certain embodiments. Generally, the system 100 comprises a segmented plenum 104 from which pressurized conditioned air 106 (see FIG. 3) is delivered downward into the main passenger cabin through a perforated overhead panel 108 (see FIGS. 2 and 3). As seen in FIG. 2, perforations 109 defined in the overhead panel 108 may be formed in a regular, equidistantly-spaced pattern, although irregular patterns are also contemplated.

Returning to FIG. 1, a sequence of partitions 110, spaced apart 2-3 feet for example, effect the segmentation of the plenum 104 into, for example, ten sub-plenums 104i that are substantially sealed from one another and disposed axially in sequence along the length of the bus 102. As best seen in FIG. 3, in certain embodiments, the panel 108 may be spaced about 1-3 inches from a roof 112 of the bus 102, generally following the contour of the roof 112 at that spacing along its entire width from the right side 114 to the left side 116 of the bus 102, with the defined plenum 104 thereby having a corresponding consistent width of about 1-3 inches. In certain embodiments, one or more coatings (not shown) may be applied to the bottom surface of the roof 112, and within the plenum, for example in liquid form for heat and/or sound insulation for instance.

Air supply into the plenum 104 is provided by way of ductwork comprising a pair of upper main ducts 118R (right) and 118L (left) (collectively 118) that deliver air into each sub-plenum 104i through dedicated balance valves 120i. Each sub-plenum 104i may thus be supplied with air by a pair of balance valves 120i (FIG. 1), one communicating with the upper main duct 118R and one communicating with the upper main duct 118L. At initial system setup, airflow through each valve 120i is adjusted, for example, through a valve adjustment mechanism such as a slide door, damper, or the like (not shown), to balance air pressure in the sub-plenums 104i so that they are able to evenly deliver, through the perforated overhead panel 108, a high volume, low turbulence blanket of air (vertically) downwards into the cabin. Importantly, the inflow of air into the cabin through system 100 is substantially or entirely by way of the plenum 104 and the perforated panel 108, without contributions from the side of the vehicle. This results in an even, unagitated downflow of the air in the cabin, avoiding vortices and mixing of the air and its contents.

Upper main ducts 118R and 118L may be made of plastic or other suitable materials. Inline booster fans 123 (FIG. 1) may be provided in the ductwork, for example in upper main ducts 118R and 118L, to aid in movement of air through the ducts and provide air circulation. In certain embodiments the fans 123 may use brushless fan motors (not shown).

With reference to FIG. 3, the ductwork of system 100 also comprises a pair of lower main ducts 122R (right) and 122L (left) (collectively 122) respectively in communication with upper main ducts 118R and 118L by way of vertical ducts 124R and 124L (collectively 124). Vertical ducts 124 may be made of plastic and generally run vertically up the back of the vehicle, in the back right and left corners thereof, to connect the respective upper main ducts 118 and lower main ducts 122. In certain embodiments, air circulation in the ductwork is facilitated by the inline booster fans 123 (FIG. 1), which aid in moving the air through the circuit, and sufficiently pressurize the plenum 104 to motivate the forced downward motion of air through the panel 108 as described above. The inline booster fans 123 may be powered by brushless motors (not shown) in some examples.

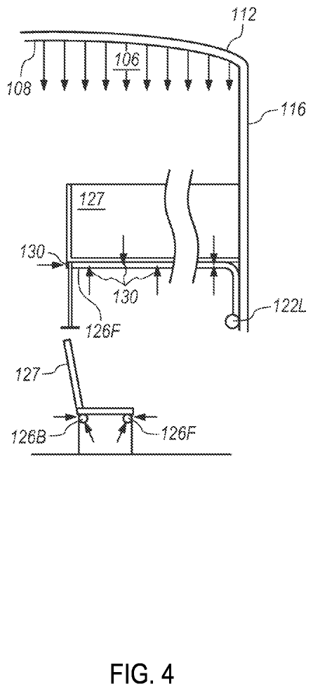

Lower main ducts 122 may also be made of plastic or other suitable material and generally extend along the floor of the vehicle cabin, receiving air from a series of seat return ducts 126. In certain embodiments, and as best illustrated in FIGS. 3 and 4, two seat return ducts 126 are associated with each seat 127 of the vehicle—a front seat return duct 126F extending along the width of and beneath the seat at the front thereof, and a back seat return duct 126B extending along the width of and beneath the seat at the back thereof. The seat return ducts 126 have inlets 129 at the aisle ends thereof (the aisle is defined as the central space between the two rows of seats on either side of the vehicle), as well as at other intermediate locations 131 along their length and directed at different angles along their perimeter (or circumference in the case of tubular cross-sections). The inlets 129 are configured to receive cabin air descending vertically from the plenum 104, permitting the air to be pulled down vertically directly in substantially all areas especially in front of the occupants, which vastly reduces lateral air motion and eliminates pathogen transfer throughout the air system of the cabin.

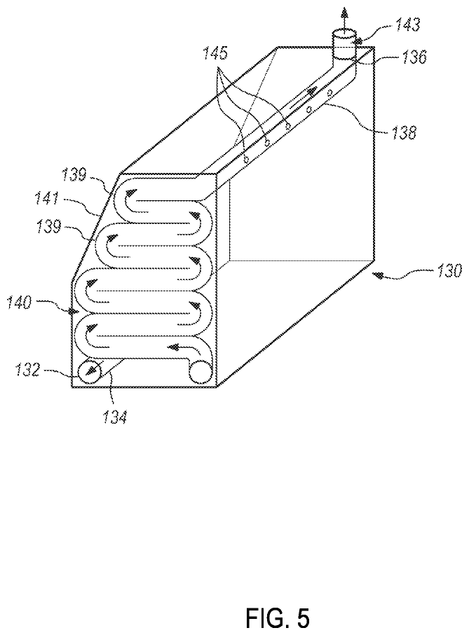

With reference to FIG. 5, in certain embodiments, system 100 includes air processing units APU 130R and APU 130L (collectively 130) through which air is circulated, for example for sterilization using ultraviolet light, and/or for heating or cooling as detailed below. Although two APUs 130 are described, fewer (or more) APUs are also possible in the system 100. APUs 130 include an air inlet 132 in communication with an inlet pipe 134 (e.g. 3-inch pipe) at one end, and an air outlet 136 in communication with an outlet pipe 138 at another end. Air is provided to the APUs 130 at the air inlet 132 from the lower main ducts 122 (FIGS. 1 and 3). The pipes 134, 138 extend substantially interiorly of the APU and communicate with a manifold 140 that is configured with suitable baffles and the like to permit the air to be sufficiently exposed to Ultraviolet-C (UVC) light from one or more UVC bulbs 139 to effectively sterilize it and kill pathogens, such as the Covid-19 virus, during passage of the air through the APU 130. The wavelengths of the light may be specifically selected to target particular pathogens, such as Covid-19, and the number of bulbs and intensity is selected for the required exposure given a particular flow rate of air through the APU. Access to the interior of the APU 130, for example to permit replacement and servicing of UVC bulbs 139, can be facilitated by a providing a removable or openable front or back panels 141. One or more such panels can be transparent to permit viewing for maintenance. Suitable circuit precautions, such as a circuit interrupter (not shown), can also be provided for additional safety.

The pipes 134, 138 in APUs 130 are provided with perforations 145 through which the air passes into or out of the manifold 140, and the perforations can have progressively increasing or decreasing diameters to balance the air pressure so that the air front traversing the manifold 140 is equally distributed and balanced therein. In certain embodiments, one or more fans 143 may be provided proximal the outlet 136, or at other locations, to help draw the air through the APU 130 and facilitate circulation in the system 100.

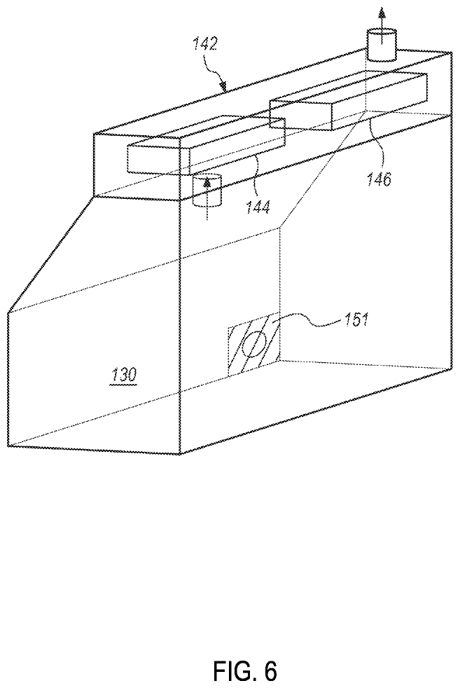

With reference to FIG. 6, in certain embodiments, one or both APUs 130 can each include a heat exchanger module 142, which can house a heater 144 and/or a cooler 146 therein. The heater 144 can operate by way of a heat exchange element (not shown) such as a hot water coil from the bus heater or powered by a standard 12 V DC (direct current) connection. The cooler 146 can also be a DC system and operate by way of an evaporator coil (not shown) coupled to an engine mounted air compressor (not shown). A slide-in air filter bank 151 can be provided in the APU 130 to help remove particulates and clean the circulating air.

It should be noted that the APUs 130 are not limited to use in the school bus 102 (FIG. 1), but rather in any multi-passenger vehicle system including, but not limited to, personal automobiles, ride share automobiles, vehicles for hire (e.g., taxis, Uber®, Lyft®, etc.), police cruisers, ambulances, or any combination thereof. In such embodiments, the APUs 130 may be located in the trunk or other suitable storage area of the multi-passenger vehicle system.

Returning to FIG. 1, in certain embodiments, an air door 152 can be provided, in air communication with the system 100, for added security and protection against introduction of pathogens such as Covid-19. The air door 152 operates to further isolate sterilized cabin air from outside air that may be contaminated.

### HVLT Ventilation Systems for Buildings

Also described herein are ventilation systems for interior spaces, such as rooms within buildings, floors of buildings, elevator carriages in elevator systems, and localized spaces within larger spaces, and other types of confined spaces, etc. As with the systems described above, the systems hereinafter described are operable to induce a high volume of air into the interior space with little or no turbulence, thereby resulting in an even, unagitated downflow (directed vertically downward) of the air in the interior space without vortices and mixing of the air and its contents. This, in turn, provides a more sanitary breathing environment for the occupants of the interior space, as such vortices and mixing within the interior space would otherwise stir up and agitate pathogens (or germs, allergens, or other undesirable particulate matter) within the interior space. The presently disclosed systems using HVLT air flow functions to inhibit formation of such vortices and mixing, such that any pathogens or germs within the interior space are drawn down towards the floor and removed from the room such that they are less likely to be inhaled by the occupants of the interior space. HVLT ventilation systems may be particularly advantageous in buildings such as schools and hospitals, but could be implemented in any type of building where enhanced air ventilation and filtration is desired.

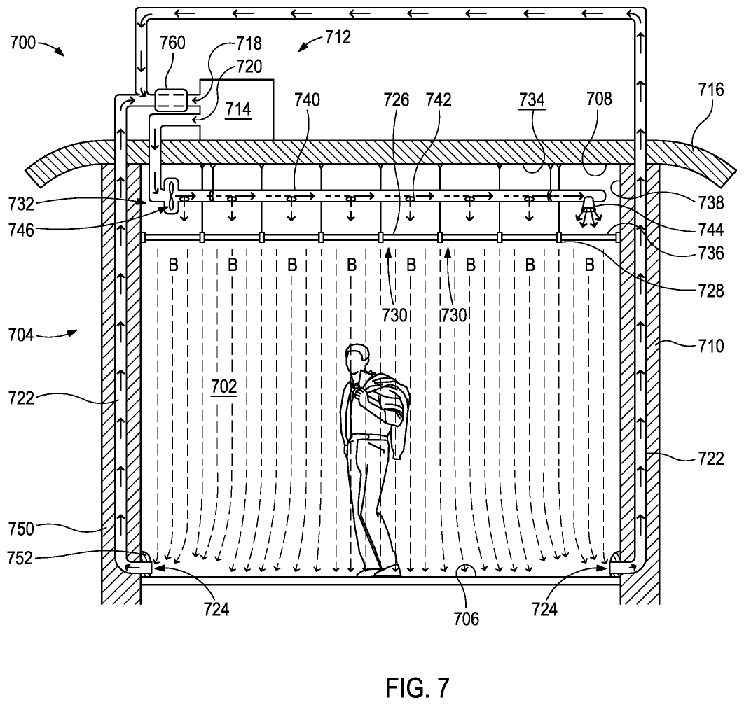

FIG. 7 schematically depicts an example ventilation system 700 for use in an interior space 702, according to one or more embodiments of the present disclosure. In the illustrated embodiment, the interior space 702 is defined within a building 704. In the illustrated example, the interior space 702 comprises a single room and interior confined space within the building 704. The building 704 may comprise a multi-room building having separate rooms, and the interior space 702 may represent the space of one of the separate rooms. The building 702 may further comprise a single story building, but could alternatively comprise a multi-story building.

As illustrated, the building 704 includes a floor 706 at a lower end of the building 704, a ceiling 708 at an upper end of the building 704 that is opposite the floor 706, and walls 710 extending between the floor 706 and the ceiling 708. In other embodiments, however, the building 704 may be differently designed.

The building 704 may further include a ventilation system 700 (hereinafter, “the system 700”), which includes an air handling unit 712 operable to induce movement of air through the interior space 702. The air handling unit 712 may include a variety of separate components for purifying, conditioning, heating, filtering, sterilizing, controlling humidity, renewing, etc., as well as circulating air through the interior space 702. To accomplish some of these tasks, the air handling unit 712 may include a blower 714. In some embodiments, the air handling unit 712 and the blower 714 may be provided on a roof 716 of the building 704. However, the air handling unit 712 and the blower 714 may alternatively be provided elsewhere relative to the building 704, for example, within a plenum defined in the interior space 702, as detailed below.

The air handling unit 712 further includes a return air inlet 718 and a supply air outlet 720. The blower 714 discharges (outputs, pumps, etc.) air out of the air handling unit 712 via the supply air outlet 720, and the air handling unit 712 receives (as an input) air via the return air inlet 718. As hereinafter described, the air handling unit 712 is operable to circulate air out of the supply air outlet 720, through the interior space 702, and back into the air handling unit 712 via the return air inlet 718.

The system 700 includes ductwork for distributing and directing air flow to and from the interior space 702. In the illustrated embodiment, the system 700 includes one or more return ducts 722 in fluid communication with the return air inlet 718. In at least one embodiment, an individual or discrete return duct 722 may be dedicated to each wall 710 of the building 704, and each discrete return duct 722 may fluidly communicate with the blower 714 via the return air inlet 718. Each return duct 722 may include one or more return openings 724 located at or near the floor 706 of the interior space 702. In some embodiments, the return opening 724 may comprise a plurality of return openings 724 that cooperatively feed a common, discrete return duct 722. In other embodiments, or in addition thereto, the return opening 724 may comprise one or more elongate slots provided at or near the floor 706 that feed a common, discrete return duct 722.

Accordingly, in at least one embodiment, each wall 710 may include its own dedicated return duct 722, which will be fed with air from corresponding return openings 724 provided at or near the floor 706 of the corresponding wall 710. In such applications, the entire perimeter of the interior space 702 will be configured to draw in air from the interior space 702 at or near the floor 706 to be conveyed to the blower 714. In larger rooms, it is contemplated to include multiple return ducts 722 running vertically up each wall 710, such as every 25 feet or 50 feet along the wall 710. In at least one embodiment, a tubular manifold may extend about the entire perimeter of the interior space 702 at or near the floor 706, and the return openings 724 provided on each wall 710 may feed air into the tubular manifold, which conveys the air into one or more return ducts 722.

In example operation, air enters the return opening 724 of the return duct 722 after having fallen down through the interior space 702 in order to be recirculated through the air handling unit 712. Regardless of how many return ducts 722 and return openings 724 are included, the return openings 724 may be provided and otherwise positioned in proximity to the floor 706. In this manner, the return openings 724 will be beneath shoulder level (and thus beneath head/mouth level) of the occupants of the interior space 702 regardless of whether the occupant is standing, sitting, or laying on a surface (such as a bed). As described herein, the air falling (circulating) through the interior space 702 will fall substantially vertically as a high volume, uniform blanket of air (depicted by reference character B and the dashed downwardly directed arrows in FIG. 7) with no (or minor) turbulence. Positioning the return openings 724 as close to the floor 706 as possible maintains the uniform flow of the air blanket B long enough such that the air flows past the occupants' mouth and nose before potentially diverging from the uniform downward flow, and outward towards the return openings 724. In embodiments, the return openings 724 are positioned at most four (4) feet above the floor 706, or at most three (3) feet above the floor 706, or at most two (2) feet above the floor 706, or at most one (1) foot above the floor 706, or bordering the surface of the floor 706.

In addition, the system 700 includes at least one perforated panel 726 arranged or suspended beneath the ceiling 708 of the interior space 702. The perforated panel 726 may be the same as the overhead panel 108 with perforations 109, as described above with reference to FIG. 2. In some embodiments, the perforated panel 726 may comprise a plurality of panels arranged together so as to substantially cover the interior space 702 (i.e., to cover the footprint of the interior space 702 at the ceiling 708). In other embodiments, however, the perforated panel 726 may be manufactured with a size that is substantially equal to a footprint of the interior space 702, such that just one perforated panels 726 is needed.

Regardless of how many perforated panels 726 are utilized, they may be suspended from the ceiling 708, above the floor 706, via a drop ceiling frame 728. In the illustrated embodiment, the drop ceiling frame 728 is a grid structure that defines a plurality of openings 730, and a corresponding one of the perforated panels 726 is arranged within an associated opening 730 in the drop ceiling frame 728.

The system 700 also includes a plenum 732 that receives air from the air handling unit 712 and is defined between the ceiling 708 and the perforated panel(s) 726. More specifically, the plenum 732 is a space defined between a lower facing surface 734 of the ceiling 708, an upper facing surface 736 of the perforated panel(s) 726, and an inner facing surface 738 (or exposed surface 738) of the walls 710. However, the plenum 732 may be differently defined as detailed herein. The air handling unit 712 is configured to convey (distribute) air into the plenum 732 to pressurize the plenum 732 such that air flow is induced through the perforated panel(s) 726 as a high volume and low turbulence blanket of air B that descends through the interior space 702. The air circulating through the interior space 702 is subsequently received by the return opening 724 in the return duct 722 for recirculation through the air handling unit 712.

In the illustrated embodiment, the system 700 includes a supply duct 740 in communication with and otherwise extending from the supply air outlet 720. The plenum 732 receives air via the supply duct 740, which extends at least partially within or through the plenum 732 for distributing air within the plenum 732. The supply duct 740 includes at least one opening 742 through which air may exit the supply duct 740 to be received within the plenum 732. In the illustrated embodiment, the supply duct 740 includes a plurality of openings 742 spaced and distributed along the supply duct 740 so as to facility an even distribution of air within the plenum 732, which in turn helps facilitate even pressurization of the plurality of panels 726.

In one or more embodiments, a louver 744 may be provided on (secured to) one or more of the openings 742 to affect distribution and flow path of the air as it is supplied to (discharged into) the plenum 732. For example, the louver 744 may be configured to direct the flow of air towards a certain area/region within the plenum 732 to facilitate even distribution/pressurization of air therein.

In the illustrated embodiment, the system 700 further includes a booster fan 746 to help facilitate pressurization of the plenum 732. Here, the booster fan 746 is arranged within the supply duct 740. In embodiments that include multiple supply ducts 740, each supply duct 740 may include a booster fan 746. Booster fans 746 may prove advantageous in applications where excessive resistance is encountered due to system configurations. Example applications where a booster fan 746 may be advantageous include long hallways or large rooms, where one or more booster fans 746 may be needed to maintain a minimum cubic foot per minute (CFM) of air flow required to keep the plenum 732 properly and equally pressurized.

In the illustrated embodiment, the supply duct 740 is a single branch of duct work extending along a length/width of the interior space 702. However, the supply duct 740 may include a plurality of ducts each running/spanning the length/width of the interior space 702, with each of the plurality of ducts evenly distributed side by side, so as to foster even distribution/pressurization of air therein. In some embodiments, the supply duct 740 may include or provide a plurality of branches that extend through the plenum 732 in a manner that facilitates even distribution/pressurization of air therein.

In some embodiments, as illustrated, the supply duct 740 physically extends through the plenum 732. In other embodiments, however, the supply duct 740 need not physically extend through the plenum 732, but instead may extend outside of the plenum 732 but with the openings 742 being in fluid communication with the plenum 732. For example, the supply duct 740 may be arranged on top of the roof 716, and holes may be formed in the roof that correspond with and are in fluid communication with the openings 742 of the supply duct 740, such that the supply duct 740 may direct air into the plenum 742 via the holes in the roof. In some embodiments, the supply duct 740 may have branches extending at least partially through the holes in the roof 716.

The return ducts 722 extend vertically but may alternatively extend in other directions as may be desired to position the return openings 724 thereof in a desired location as described herein. In some embodiments, as illustrated, the return ducts 722 may be positioned or located within or inside the wall 710, such that the return ducts 722 extend within an interior 750 of the wall 710. In this manner, the return ducts 722 are not exposed to the occupants inside the interior space 702 or to people outside of the building 704. In other embodiments, however, one or more of the return ducts 722 may be arranged such that they extend at least partially within/through the interior space 702, as exposed conduits, with their return openings 724 exposed within the interior space 702. In the illustrated embodiment, for example, the return duct 722 may be mounted on (or to) the inner facing surface 738 of the wall 710. In other embodiments, the return ducts 722 may be located exterior to the interior space 702, such as on the other side of the wall 710. In such embodiments, the return opening 724 may be configured and located to fluidly communicate with the interior space 702. For example, the return duct 722 may be located outside of the interior space 702 (e.g., outside of the building 704) and mounted on an outside surface of the wall 710, but the return opening 724 may be in fluid communication with the interior space 702 via an opening in the wall 710.

In the illustrated embodiment, a baseboard 752 is provided at and otherwise mounted to the inner facing surface 738 of the wall 710 at the junction between the wall 710 and the floor 706. Here, one or more openings are formed in the baseboard 752 to accommodate the return opening(s) 724 in close proximity to the floor 706. Positioning the return openings 724 within the baseboards 752 ensures that the return openings 724 are located towards the bottom of the interior space 702, well beneath the mouth/nose of the occupants, which in turn ensures that the withdrawal and receiving of the air in the interior space 702 through the return openings 724 does not create turbulence that could otherwise agitate or stir up pathogens, germs, allergens, etc. to be inhaled by the occupants. In embodiments where the return opening 724 comprises an elongate slot, as discussed above, the opening 724 may be provided beneath the baseboard 752, such as in a gap between the baseboard 752 and the floor 706.

In embodiments, the system 700 further includes an air processing unit 760 that is operable to filter, sanitize, and/or sterilize air conveyed through the interior space 702. The air processing unit 760 may be the same as or similar to the air processing unit (APU) 130 described herein with respect to FIGS. 5 and 6. Accordingly, in some applications, the air processing unit 760 may be configured to sterilize air circulating through the interior space 702 using Ultraviolet-C (UVC) light. In the illustrated embodiment, the air processing unit 760 is positioned upstream of the return air inlet 718. For example, the air processing unit 760 may be provided on (within) the return duct 722. In other embodiments, the air processing unit 760 may be positioned downstream of the supply air outlet 720. For example, the air processing unit 760 may be arranged within the supply duct 740 or the plenum 732. In embodiments, the air processing unit 760 is integrated into the air handling unit 712.

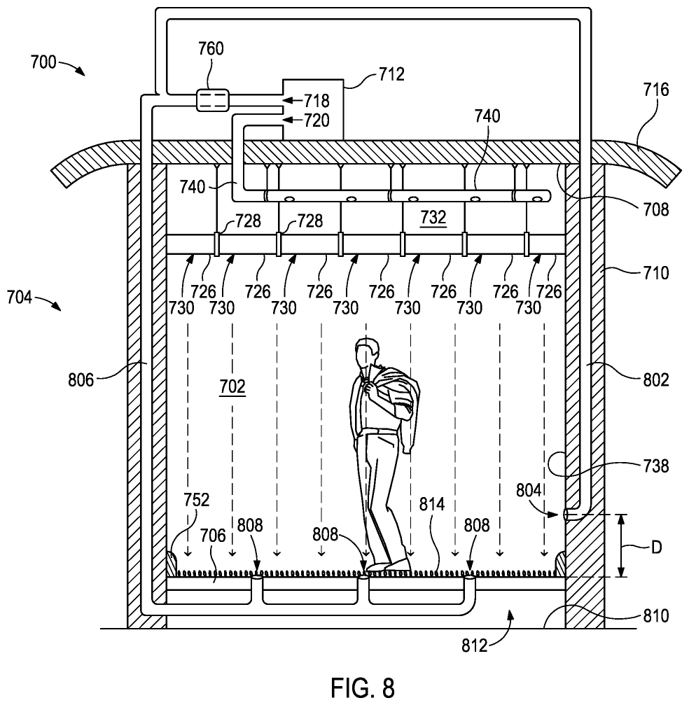

FIG. 8 depicts an alternate configuration (embodiment) of the system 700, according to alternate embodiments of the present disclosure. In particular, FIG. 8 depicts the system 700 having differently arranged and configured return ducts as compared to the return duct(s) 722 of FIG. 7. In the illustrated embodiment, the system 700 includes a first return duct 802 having at least one return opening 804 and a second return duct 806 having a plurality of return openings 808.

In the illustrated embodiment, the first return duct 802 extends vertically within the wall 710 and towards the floor 706. The return opening 804 for the first return duct 802 is arranged (defined) within the inner facing surface 738 of the wall 710, at a distance D above the floor 706. Here, the return opening 804 is also positioned above the baseboard 752. Thus, a corresponding opening is formed in the inner facing surface 738 of the wall 710 through which the first return duct 802 extends so as to position the return opening 804 within the wall 710. As with the embodiment of FIG. 8, in some embodiments, each wall 710 may include a dedicated or discrete return duct 802, and each wall 710 may include one or more return openings 804 that feed a corresponding return duct 802.

While spaced above the floor 706 by the distance D, the return opening 804 may also be located beneath the breathing level of occupants in the interior space 702. Accordingly, the distance D at which the return opening 804 is located may be beneath mouth/nose level of occupants within the interior space 702. In embodiments, the distance D is at most four (4) feet, at most three (3) feet, at most two (2) feet, or at most one (1) foot. In other embodiments, the distance D equals the height of the baseboard 752 such that the return opening 804 is positioned at or just above the junction between and top of the baseboard 752 and the wall 710. While just one first return duct 802 having a single return opening 804 is depicted, more than one may be provided, and one or more of the first return ducts 802 may fluidly communicate with two or more return openings 804 in order to optimize flow of air out of the interior space 702.

In the illustrated embodiment, the second return duct 806 extends vertically and at least partially under the upper surface of the floor 706, and the plurality of return openings 808 may be defined in or otherwise provided in the floor 706. In particular, openings are formed in the floor 706, and the second return duct 806 includes branches (individual conduits) that at least partially extend through the openings so that the return openings 808 are arranged within the floor 706. In this manner, return air is drawn back into the return duct 806 through the floor 706.

In the illustrated embodiment, a layer of carpet 814 is provided on an upper surface of the floor 706. In some embodiments, the carpet 814 may comprise porous walk-off carpet, which is breathable and allow unobstructed air to flow through the return openings 808. In other embodiments, the carpet 814 may be replaced with one or more perforated floor panels. In the illustrated embodiment, three (3) return openings 808 are depicted on the second return duct 806. However, the second return duct 806 may have more or less than three (3) return openings 808, and such return openings 808 may be positioned so as to optimize air flow out of the interior space 702. Further, while just one second return duct 806 is depicted, more than one may be utilized (having varying number of return openings 808) as may be desired to allow for desired air flow exiting the interior space 702.

In the illustrated embodiment, the floor 706 may comprise a raised floor. More specifically, the building 704 may include a base 810 that also partially defines the interior space 702, and the base 810 may be opposite the ceiling 708. The floor 706 may comprise a surface that is raised (or suspended) above the base 810 (e.g., a raised access floor), such that an underfloor void 812 is defined beneath the floor 706 and above the base 810. Here, the second return duct 806 extends at least partially through the underfloor void 812 defined between the base 810 and the floor 706. In other embodiments, however, the floor 706 may comprise the ground floor of the building 704. In such embodiments, the second return duct 806 may be provided (buried) in underlying earthen material. In one or more embodiments, the return ducts 802, 806 may be used in tandem.

In some embodiments, as with the system 700 of FIG. 7, the air processing unit 760 may be included to filter, sanitize, and/or sterilize air conveyed through the interior space 702. In the illustrated embodiment, the air processing unit 760 is positioned upstream of the return air inlet 718 and in fluid communication with the return ducts 802, 806. In other embodiments, the air processing unit 760 may be positioned downstream of the supply air outlet 720, such as within the supply duct 740 or the plenum 732.

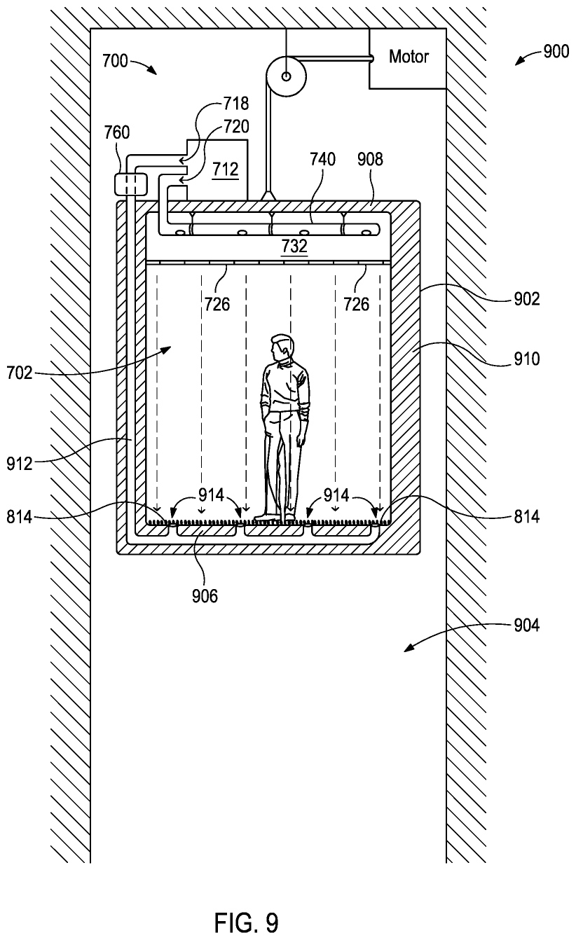

FIG. 9 depicts an embodiment where the system 700 is utilized with an elevator system 900, according to one or more embodiments. As shown, the elevator system 900 includes an elevator carriage 902 that may travel within an elevator shaft 904. The elevator carriage 902 includes a floor 906, a ceiling 908 opposite the floor 906, and walls 910 extending between the floor 906 and the ceiling 908. Also, one or more perforated panels 726 may be arranged or suspended beneath the ceiling 908, such that the plenum 732 is defined between the perforated panel(s) 726 and the ceiling 908.

The system 700 is integrated within the elevator carriage 902 and may be configured as described above with reference to FIGS. 7 and 8. Thus, the supply duct 740 extends from the air handling unit 712 into the plenum 732 to distribute air within and pressurize the plenum 732, as described above. Also, in the illustrated embodiment, the system 700 includes a return duct 912 extending from the air handling unit 712, downward through the walls 710 and within (and beneath) an upper surface of the floor 906. As illustrated, the return duct 912 includes a plurality of openings 914 that fluidly communicate with the interior space 702 through the floor 906, similar to what is described in FIG. 8. However, the return duct 912 may also or instead have openings formed in the wall 910, as described in FIG. 7. Moreover, in some embodiments, the floor 906 may include a layer of carpet 814, as generally described above, or alternatively include one or more perforated floor panels that allows air circulation between the interior space 702 and the return duct 912.

While not shown, it is contemplated herein that the elevator system 900 further include return openings provided in the walls 910 of the carriage 902, similar to the systems shown and described herein with reference to FIGS. 7 and 8. In such embodiments, the return openings provided in the walls 910 may fluidly communicate with dedicated or discrete return ducts extending within the corresponding walls 910, and may operate in conjunction with the return duct 912 and the corresponding openings 914 generally described herein.

In some embodiments, as with the system 700 of FIG. 7, the air processing unit 760 may be included to filter, sanitize, and/or sterilize air conveyed through the interior space 702. In the illustrated embodiment, the air processing unit 760 is positioned upstream of the return air inlet 718 and in fluid communication with the return duct 912. In other embodiments, the air processing unit 760 may be positioned downstream of the supply air outlet 720, such as within the supply duct 740 or the plenum 732.

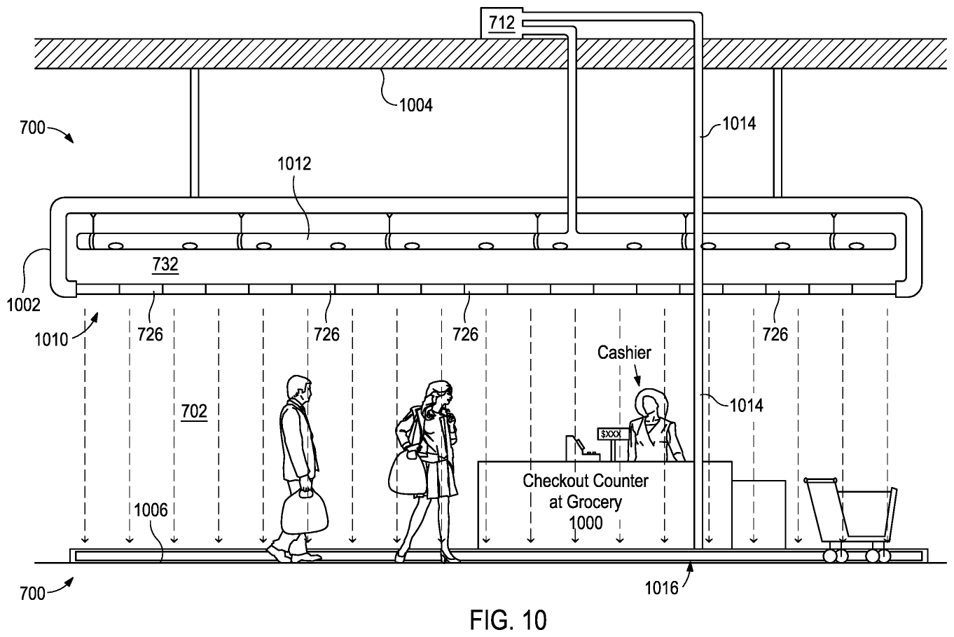

FIG. 10 depicts another example use for the system 700. In particular, FIG. 10 depicts a structure 1000, such as store checkout counter, and the system 700 may be incorporated into the structure 1000 to help drawn in air and filter and purify the drawn air. As illustrated, the interior space 702 may comprise an open area within a much larger space, such as a large retail store (e.g., a grocery store, a hardware store, etc.), and a housing 1002 is suspended from a ceiling 1004 of the large open space. Here, the structure 1000 is a checkout counter provided on a floor 1006 of the large open space. As will be appreciated, other types of structures may be mounted on the floor 1006 instead of or in addition to the checkout counter to be used with the system 700.

As shown, the housing 1002 includes a bottom facing opening 1010 within which one or more perforated panels 726 are arranged. Also, the plenum 732 is defined within the housing 1002 in conjunction with the perforated panel(s) 726. A supply duct 1012 extends from the air handling unit 712 to the plenum 732 to deliver air to the plenum 732, as generally described herein. Further, a return duct 1014 extends to the air handling unit 712 from the structure 1000. The return duct 1014 may be in fluid communication with one or more return openings 1016 provided at or near the floor 1006. In at least one embodiment, as illustrated, the return opening 1016 may comprise an elongate slot having a length substantially similar to the length of the bottom facing opening 1010. In such embodiments, the flow of air discharged from the perforated panel(s) 726 may drop substantially vertically through the interior space 702 until being drawn into the return duct 1014 via the elongated slot of the return opening 1016. In other embodiments, however, the return opening 1016 may comprise a plurality of return openings spaced from each other at or near the floor 1006 to receive the air discharged from the perforated panel(s) 726.

Described herein is a method of ventilating an interior space, for example, utilizing the system 700 described above. In embodiments, the method includes conveying air from an air handling unit into a plenum, wherein the plenum is defined between a ceiling of the interior space and at least one perforated panel arranged beneath the ceiling of the interior space. The method may further include inducing air flow is evenly and uniformly through the at least on perforated panel as a high volume and low turbulence blanket of air that descends through the interior space towards a floor of the interior space. Also, the method may include drawing in air from the interior space into one or more return ducts via a return opening in the one or more return ducts that is disposed proximate to the floor, and recirculating the air received by the one or more return ducts back through the air unit system.

In embodiments, the method may further include increasing pressurization of the air within the plenum via a booster fan. In embodiments, the method may further include sterilizing the air via UVC light.

Embodiments disclosed herein include:
A. A ventilation system for an interior space includes an air handling unit configured to circulate air through the interior space, the air handling unit having a return air inlet and a supply air outlet, a return duct in fluid communication with the return air inlet and having a return opening proximate to a floor of the interior space, and at least one perforated panel arranged beneath a ceiling of the interior space and thereby defining a plenum between the ceiling and the at least one perforated panel, wherein the supply air outlet is in fluid communication with the plenum to provide air into the plenum and thereby pressurize the plenum such that air flow is induced through the at least one perforated panel as a high volume, low turbulence blanket of air that descends through the interior space and is received by the return opening in the return duct for recirculation through the air handling unit.
B. A method for ventilating an interior space, the method including the steps of conveying air from an air handling unit into a plenum defined between a ceiling of the interior space and at least one perforated panel arranged beneath the ceiling of the interior space, pressurizing the air within the plenum and forcing the air through the at least one perforated panel evenly and uniformly as a high volume, low turbulence blanket of air that descends through the interior space and towards a floor of the interior space, drawing in air from the interior space into one or more return ducts via a return opening disposed proximate to the floor, and recirculating the air received by the one or more return ducts back through the air handling unit.
C. A ventilation system for an interior space includes a housing suspended from a ceiling of the interior space, a structure provided on a floor of the interior space, at least one perforated panel arranged within a bottom facing opening of the housing, a plenum defined between an interior of the housing and the at least one perforated panel, an air handling unit configured to circulate air through the interior space, the air handling unit having a return air inlet and a supply air outlet, and a return duct in fluid communication with the return air inlet and extending to the structure, the return duct having a return opening provided in the structure proximate to the floor, wherein the supply air outlet is in fluid communication with the plenum to provide air into the plenum and thereby pressurize the plenum such that air flow is induced through the at least one perforated panel as a high volume, low turbulence blanket of air that descends through the interior space, beneath the housing, and is received by the return opening in the return duct for recirculation through the air handling unit.

Each of embodiments A, B, and C may have one or more of the following additional elements in any combination: Element 1: further comprising a supply duct in fluid communication with the supply air outlet and extending into the plenum to distribute the air within the plenum. Element 2: wherein the supply duct includes a plurality of openings that evenly distribute the air within the plenum and thereby facilitate uniform pressurization of the plenum. Element 3: further comprising a louver on at least one of the at openings. Element 4: wherein the interior space includes a wall extending between the ceiling and the floor, and wherein the return duct is arranged within or behind the wall and the return opening is defined in the wall. Element 5: further comprising a baseboard provided on an inner facing surface of the wall at the floor, wherein the return opening is defined through the baseboard. Element 6: wherein the return duct is arranged within the interior space. Element 7: wherein the return duct extends beneath the floor and fluidly communicates with the interior space through the floor. Element 8: wherein the interior space is defined by a base that is opposite the ceiling, and the floor comprises a surface raised above the base to thereby define an underfloor void between the base and the floor, and wherein the return duct extends into the underfloor void. Element 9: further comprising carpet provided on an upper surface of the floor. Element 10: wherein the at least one perforated panel is suspended from the ceiling via a drop ceiling frame. Element 11: wherein the interior space is defined within a building. Element 12: wherein the interior space is defined within an elevator carriage. Element 13: further comprising a booster fan to pressurize the plenum. Element 14: further comprising an air processing unit in fluid communication with the air handling unit and operable to sterilize air conveyed through the interior space.

Element 15: further comprising increasing a pressure of the air within the plenum with a booster fan. Element 16: further comprising sterilizing the air with an air processing unit in fluid communication with the air handling unit.

Element 17: wherein the interior space is defined within a store building and the structure comprises a checkout counter.

By way of non-limiting example, exemplary combinations applicable to A, B, and C include: Element 1 with Element 2; Element 2 with Element 3; Element 4 with Element 5; Element 7 with Element 8; and Element 7 with Element 9.

While embodiments and applications have been shown and described, it would be apparent to those skilled in the art having the benefit of this disclosure that many more modifications than mentioned above are possible without departing from the inventive concepts disclosed herein. The invention, therefore, is not to be restricted based on the foregoing description. This disclosure encompasses all changes, substitutions, variations, alterations, and modifications to the example embodiments herein that a person having ordinary skill in the art would comprehend. Similarly, where appropriate, the appended claims encompass all changes, substitutions, variations, alterations, and modifications to the example embodiments herein that a person having ordinary skill in the art would comprehend. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

What is claimed is:

1. A ventilation system for an interior space, comprising:
an air handling unit configured to circulate air through the interior space, the air handling unit having a return air inlet and a supply air outlet;
a return duct in fluid communication with the return air inlet and having a return opening proximate to a floor of the interior space; and
at least one perforated panel arranged beneath a ceiling of the interior space and thereby defining a plenum between the ceiling and the at least one perforated panel,
wherein the supply air outlet is in fluid communication with the plenum to provide air into the plenum and thereby pressurize the plenum such that air flow is induced through the at least one perforated panel as a high volume, low turbulence blanket of air that descends through the interior space and is received by the return opening in the return duct for recirculation through the air handling unit.

2. The ventilation system of claim 1, further comprising a supply duct in fluid communication with the supply air outlet and extending into the plenum to distribute the air within the plenum.

3. The ventilation system of claim 2, wherein the supply duct includes a plurality of openings that evenly distribute the air within the plenum and thereby facilitate uniform pressurization of the plenum.

4. The ventilation system of claim 3, further comprising a louver on at least one of the at openings.

5. The ventilation system of claim 1, wherein the interior space includes a wall extending between the ceiling and the floor, and wherein the return duct is arranged within or behind the wall and the return opening is defined in the wall.

6. The ventilation system of claim 5, further comprising a baseboard provided on an inner facing surface of the wall at the floor, wherein the return opening is defined through the baseboard.

7. The ventilation system of claim 1, wherein the return duct is arranged within the interior space.

8. The ventilation system of claim 1, wherein the return duct extends beneath the floor and fluidly communicates with the interior space through the floor.

9. The ventilation system of claim 8, wherein the interior space is defined by a base that is opposite the ceiling, and the floor comprises a surface raised above the base to thereby define an underfloor void between the base and the floor, and wherein the return duct extends into the underfloor void.

10. The ventilation system of claim 8, further comprising carpet provided on an upper surface of the floor.

11. The ventilation system of claim 1, wherein the at least one perforated panel is suspended from the ceiling via a drop ceiling frame.

12. The ventilation system of claim 1, wherein the interior space is defined within a building.

13. The ventilation system of claim 1, wherein the interior space is defined within an elevator carriage.

14. The ventilation system of claim 1, further comprising a booster fan to pressurize the plenum.

15. The ventilation system of claim 1, further comprising an air processing unit in fluid communication with the air handling unit and operable to sterilize air conveyed through the interior space.

16. A method for ventilating an interior space, comprising:
conveying air from an air handling unit into a plenum defined between a ceiling of the interior space and at least one perforated panel arranged beneath the ceiling of the interior space;
pressurizing the air within the plenum and forcing the air through the at least one perforated panel evenly and uniformly as a high volume, low turbulence blanket of air that descends through the interior space and towards a floor of the interior space;
drawing in air from the interior space into one or more return ducts via a return opening disposed proximate to the floor; and
recirculating the air received by the one or more return ducts back through the air handling unit.

17. The method of claim 16, further comprising increasing a pressure of the air within the plenum with a booster fan.

18. The method of claim 16, further comprising sterilizing the air with an air processing unit in fluid communication with the air handling unit.

19. A ventilation system for an interior space, comprising:
a housing suspended from a ceiling of the interior space;
a structure provided on a floor of the interior space;
at least one perforated panel arranged within a bottom facing opening of the housing;
a plenum defined between an interior of the housing and the at least one perforated panel;
an air handling unit configured to circulate air through the interior space, the air handling unit having a return air inlet and a supply air outlet; and
a return duct in fluid communication with the return air inlet and extending to the structure, the return duct having a return opening provided in the structure proximate to the floor,
wherein the supply air outlet is in fluid communication with the plenum to provide air into the plenum and thereby pressurize the plenum such that air flow is induced through the at least one perforated panel as a high volume, low turbulence blanket of air that descends through the interior space, beneath the housing, and is received by the return opening in the return duct for recirculation through the air handling unit.

20. The ventilation system of claim 19, wherein the interior space is defined within a store building and the structure comprises a checkout counter.
